(19) United States
(12) Patent Application Publication
(10) Pub. No.: US 2026/0315334 A1
(43) Pub. Date: Oct. 15, 2026

(54) MUCOADHESIVE COMPOSITIONS COMPRISING DI-VALENT CATION DONOR, METHOD OF CURING THE SAME

(71) Applicant: ISP INVESTMENTS LLC, Wilmington, DE (US)

(72) Inventors: Sounak SARKAR, Hillsborough, NJ (US); Amit GUPTA, Mumbai, Maharashtra (IN); Philip John OTHS, Mendham, NJ (US); Petros GEBRESELASSIE, Whitehouse Station, NJ (US); Hani M. FARES, Somerset, NJ (US)

(73) Assignee: ISP INVESTMENTS LLC, Wilmington, DE (US)

(21) Appl. No.: 19/483,734

(22) PCT Filed: May 10, 2024
(86) PCT No.: PCT/US2024/028787

§ 371 Date: Nov. 12, 2025

Related U.S. Application Data
(60) Provisional application No. 63/501,720, filed on May 12, 2023.

(51) Int. Cl. A61K 6/30; A61K 6/60; A61K 6/70

(52) U.S. Cl. CPC A61K 6/30; A61K 6/60; A61K 6/70

(57) ABSTRACT

The present invention provides a mucoadhesive composition comprising: (a) non-neutralized diacidic form of lower alkyl vinyl ether-maleic acid copolymer, (b) sodium carboxymethylcellulose (Na-CMC) polymer, (c) water-insoluble, water-swellable, cross-linked polyvinylpyrrolidone (PVPP) polymer, (d) at least one di-valent (M⁺⁺) cation donor, (e) at least one neutralizer, and (f) at least one oral care ingredient. Further, the present invention also provides a method of in situ curing of mucoadhesive composition of claimed invention at the application site.

The presently disclosed process(es), procedure(s), method(s), product(s), result(s), and/or concept(s) (collectively referred to hereinafter as the “present application, present disclosure or invention”) relates generally to a mucoadhesive composition with a di-valent cation donor and a neutralizer.

### BACKGROUND OF THE INVENTION

This invention relates to a mucoadhesive composition with a di-valent cation donor and a neutralizer.

mucoadhesive compositions, including denture fixative or denture adhesive formulations, available generally in the form of creams, powders, or extruded dry wafers/films, are used to provide secure hold of artificial dentures in mouth for users of partial or full dentures or deliver therapeutic actives to desired areas inside buccal cavity by topical applications. Typically, mucoadhesive composition including denture adhesive cream consists of water soluble or water swellable adhesive polymers suspended in an orally acceptable carrier matrix generally composed of different proportions of long chain hydrocarbons or long chain fatty acids like mineral oils, vegetable oils, petrolatum, and the like. During use, activation of the adhesive components in mucoadhesive composition, particularly denture adhesive cream occurs on contact with saliva, resulting in hydration, swelling and subsequent buildup of adhesive and cohesive hold of the denture adhesive formulation applied between the gum and the denture. Duration and strength of adhesive hold provided by a denture adhesive formulation is the primary quantitative performance parameter of the denture adhesive. Other semi-quantitative or qualitative parameters that are used to define overall performance characteristics of a denture adhesive formulation might include film thickness and cushioning effect provided by the denture adhesive between gum and denture, preventing food particles from entering interstitial space between gum and denture by providing superior gap sealing and loss of denture adhesive material from between gum and denture due to oozing during its time of use. Since common mucoadhesive composition including denture adhesive creams are essentially suspensions or dispersions of solid active ingredients in a semi-solid or liquid matrix composed of various kinds of oils, loss of shelf life stability of the formulation due to syneresis and separation of the carrier matrix and solid active ingredients might also adversely affect adhesion performance.

Thus, an ideal mucoadhesive composition, particularly denture fixative cream should have suitable shelf-life stability over a sustained period of time, provide sufficiently high instant dry and wet tack and prolonged adhesive hold during use, provide adequate cushioning between gum and denture to ensure comfort of use, be able to seal gaps between denture and gum to prevent food particles from entering the gap, have reduced uncomfortable mouth feel due to oozing out of partially hydrated denture adhesive from between gum and denture and easy cleanability after use.

U.S. Pub. No. 20220401395A1 (assigned to ISP Investments LLC) describes a mucoadhesive composition including denture adhesive composition comprising: (i) 10 to about 75 wt. % of a maleic acid or anhydride copolymer; (ii) 10 to about 50 wt. % of a cellulose ether; (iii) 0.1 to 10 wt. % of a crosslinked polyvinyl pyrrolidone that is swellable but not soluble in water; and (iv) 30 to 70 wt. % of an orally acceptable carrier based on the total weight of the composition. Also disclosed is a method of use thereof and a process for preparing the same.

JP. Pat. No. 06908965B2 (assigned to Kobayashi Pharmaceutical Co., Ltd.) discloses a denture stabilizer comprises the following (A) and (B) components: (A) at least one adhesive component selected from the group consisting of a lower alkylvinyl ether/maleic anhydride copolymer, a derivative thereof, and salt thereof and (B) hydrogen phosphate.

PCT. Pub. No. WO199210988A1 (assigned to Richardson-Vicks, Inc.) discloses adhesives and stabilizer compositions comprising a lower alkyl vinyl ether-maleic acid copolymer and their salts, said copolymer having a specific viscosity larger than 1.2, the specific viscosity being determined in methyl ethyl ketone at 25° C. and wherein said copolymer has a bulk density from about 0.3 to about 1.2 grams per cubic centimeter and a specific surface area from about 0.5 to about 2.5 square meters per gram.

In view of the foregoing, still there is a need for an ideal mucoadhesive composition having an advantage of controlled dissolution and activation of primary adhesive polymer giving an extended duration of adhesive/cohesive hold, and a controlled ooze.

Surprisingly, it was found that an in situ generation of di-valent (M⁺⁺) cation by hydration of di-valent (M⁺⁺) cation donor enables cross-linking and forms a stronger interpenetrating network of lower alkyl vinyl ether-maleic acid copolymer and sodium carboxymethylcellulose with improved adhesion and/or cohesion at significantly lower levels of alkyl vinyl ether-maleic acid copolymer, and further provides a better ooze control with water-insoluble, water-swellable, cross-linked polyvinylpyrrolidone (PVPP).

### SUMMARY OF THE INVENTION

One aspect of the present application is to provide a mucoadhesive composition, comprising: (a) from about 2 to about 60 wt. % of non-neutralized diacidic form of lower alkyl vinyl ether-maleic acid copolymer, (b) from about 5 to about 60 wt. % of a sodium carboxymethylcellulose (Na-CMC) polymer, (c) from about 0.5 to about 10 wt. % of water-insoluble, water-swellable, cross-linked polyvinylpyrrolidone (PVPP) polymer, (d) from about 0.5 to about 25 wt. % of at least one di-valent (M⁺⁺) cation donor, (e) from about 0.5 to about 25 wt. % of at least one neutralizer, and (f) from about 0.001 to about 90 wt. % of at least one orally acceptable carrier or oral care ingredient or both.

Another aspect of the present application is to provide a method of in situ curing of mucoadhesive composition comprising: (a) from about 2 to about 60 wt. % of non-neutralized diacidic form of lower alkyl vinyl ether-maleic acid copolymer, (b) from about 5 to about 60 wt. % of a sodium carboxymethylcellulose (Na-CMC) polymer, (c) from about 0.5 to about 10 wt. % of water-insoluble, water-swellable, cross-linked polyvinylpyrrolidone (PVPP) polymer, (d) from about 0.5 to about 25 wt. % of at least one di-valent (M⁺⁺) cation donor, (e) from about 0.5 to about 25 wt. % of at least one neutralizer, and (f) from about 0.001 to about 90 wt. % of at least one orally acceptable carrier or oral care ingredient or both; the method comprising the steps of: (a) providing from about 0.01 to about 99.9 wt. % of mucoadhesive composition at the application site, (b) releasing di-valent (M⁺⁺) cation in situ by hydration of di-valent (M⁺⁺) cation donor in the presence of or by introduction of a hydrating agent, (c) enabling di-valent cation mediated cross-linking of the lower alkyl vinyl ether-maleic acid copolymer, (d) forming an interpenetrating polymer network (IPN) of lower alkyl vinyl ether-maleic acid copolymer and carboxymethylcellulose, and (e) obtaining an in situ activated and cured mucoadhesive composition.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a”, “an”, and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached.

In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

The term “polymer” refers to a compound comprising repeating structural units (monomers) connected by covalent chemical bonds. Polymers may be further derivatized, cross-linked, grafted or end-capped Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The terms “denture fixative” or “mucoadhesive” or “denture adhesive” refer to an adhesive that sticks to or adheres to the skin or mucus or mucosal cell or dental surfaces.

As used herein, “denture” refers to either partial or full upper or lower denture, or all of the above. Desirably, composition should function as an effective means for insulating, cushioning and securely positioning the denture. The composition should retain its characteristics and properties in the powder and cream forms during storage under various climatic conditions, such as temperature and humidity; be readily and easily capable of application to the denture surface; not be irritating or uncomfortable to the user; be safe and nontoxic; have no disagreeable odor or color; have no unpalatable taste; should not facilitate growth of harmful organisms or microbes usually found in mouth; and should not facilitate putrefaction: or malodorous decomposition of foods or secretions lodging beneath or adjacent to the denture.

As used herein, “diacidic form of lower alkyl vinyl ether-maleic acid copolymer” refers to a predetermined weight average molecular weight of from about 500,000 to 3,000,000 suitable for denture adhesives made by: copolymerizing about 50 mole percent of maleic anhydride, about 50 mole percent of a C₁-C₄ alkyl vinyl ether, in the presence of a free radical initiator, at about 50° C. to 150° C. with solvent or solvent-free process to produce as a uniform, fine powder having substantially no residual maleic anhydride.

The alkyl vinyl ether maleic anhydride copolymers are obtained by co-polymerizing an alkyl vinyl ether monomer, such as methyl vinyl ether, ethyl vinyl ether, divinyl ether, propyl vinyl ether and isobutyl vinyl ether, with maleic anhydride to yield the corresponding alkyl vinyl ether-maleic anhydride copolymer which is readily hydrolysable to the acid copolymer. Both anhydride and acid forms are also available from commercial suppliers. For example, Ashland Inc. provides both the polymeric free acid form and the corresponding anhydride form under its “GANTREZ” trademark as the “GANTREZ S Series”.

As used herein, “cellulose ether” refers to a cellulose derivative obtained by etherifying a hydroxyl group of cellulose using an etherifying agent Useful cellulose ethers can be selected from the group consisting of methylcellulose, hydroxyethylcellulose, hydroxypropyl cellulose, hydroxypropyl methylcellulose, hydrophobically modified hydroxyalkyl cellulose, sodium carboxymethylcellulose and mixtures thereof.

As used herein, the term “mono-valent cation” refers to a cation having a valency of one (M⁺). Accordingly, the term “di-valent cation” refers to a cation having a valency of two (M⁺⁺). The mono-valent cation is selected from the group consisting of sodium, potassium, lithium and combinations thereof. The mono-valent cation is sodium. The di-valent cation is selected from the group consisting of calcium, magnesium, strontium, iron, zinc, tin, copper, and combinations thereof. More preferably, the di-valent cation is calcium, magnesium, zinc, and combinations thereof.

As used herein “cross-linked or cross-linking” refers to a composition containing intra-molecular and/or inter-molecular cross-links, whether arising through covalent or non-covalent bonding. “Non-covalent” bonding includes both hydrogen bonding and electrostatic (ionic) bonding.

As used herein, the term “Interpenetrating polymer network” or “IPN” refers to a blend of two or more polymers in a network form, at least one of which is synthesized and/or crosslinked in the immediate presence of the other(s). An IPN can be distinguished from polymer blends, blocks, or grafts in two ways: (1) an IPN swells, but does not dissolve in solvents, and (2) creep and flow are suppressed.

As used herein, the term “in situ” is a specific buccal cavity site of skin or mucosa or mucosal cell or denture surfaces.

As used herein, the term “functionalized” with reference to carboxymethyl cellulose of this application refers to the presence of mono-valent and one or more di-valent cations on the carboxymethylcellulose. Various mono-valent and di-valent cations may be introduced in a carboxymethylcellulose by way of one or more functionalization reactions known to a person having ordinary skill in the art. Non-limiting examples of functionalization reactions include ion-exchange process comprising a reaction of (i) at least one mono-valent (M⁺) carboxymethyl cellulose salt(s); and (ii) one or more di-valent (M⁺⁺) metal salt(s); wherein the di-valent (M⁺⁺) salt is selected from the group consisting of M⁺⁺ chloride or M⁺⁺ sulfate or M⁺⁺ carbonate wherein the weight ratio of M⁺ to M⁺⁺ in the CMC polymer is from about 1:70 to about 70:1.

As used herein, including examples the following abbreviations are elaborated:
(i) PVP: Polyvinylpyrrolidone;
(ii) MVE: Methyl vinyl ether;
(iii) MA: Maleic anhydride/Maleic acid;
(iv) Na-CMC: Sodium Carboxymethyl cellulose;
(v) wt. %: Weight percent;
(vi) FlexiThix: Lightly to moderately crosslinked polyvinyl pyrrolidone;
(vii) Polyplasdone XL-10: Self-crosslinked polyvinyl pyrrolidone;
(viii) PVP K-90: Polyvinyl pyrrolidone powder average molecular weight of 1,300,000 in Daltons;
(ix) Aqualon CMC 7H3SXF: Sodium Carboxymethyl cellulose;
(x) PVP K-15: Polyvinylpyrrolidone powder average molecular weight of 8,000 in Daltons, and
(xi) other cellulose ethers like HEC, HPC, EC, divalent cation modified cellulose ethers.

In a non-limiting embodiment, the present application provides a mucoadhesive composition, comprising: (a) from about 2 to about 60 wt. % of non-neutralized diacidic form of lower alkyl vinyl ether-maleic acid copolymer, (b) from about 5 to about 60 wt. % of a sodium carboxymethylcellulose (Na-CMC) polymer, (c) from about 0.5 to about 10 wt. % of water-insoluble, water-swellable, cross-linked polyvinylpyrrolidone (PVPP) polymer, (d) from about 0.5 to about 25 wt. % of at least one di-valent (M++) cation donor, (e) from about 0.5 to about 25 wt. % of at least one neutralizer, and (f) from about 0.001 to about 90 wt. % of at least one orally acceptable carrier and/or oral care ingredient.

In another non-limiting embodiment, the present application provides a mucoadhesive composition, wherein the maleic acid copolymer comprises maleic acid and a lower alkyl (C₃-C₁₂) vinyl ether having a predetermined weight average molecular weight of about 500,000 to 3,000,000 suitable for denture adhesives made by copolymerizing about 50 mole percent of maleic anhydride, about 50 mole percent of a C₁-C₁₂ alkyl vinyl ether, in the presence of a free radical initiator, at about 50° to 150° C., in a solvent or solvent free process to produce a uniform, fine powder having substantially no residual maleic anhydride.

In another non-limiting embodiment, the lower alkyl group of vinyl ether-maleic acid copolymer has carbons atoms from 1 to 12 carbon atoms, from 1 to 8 carbon atoms, from 1 to 6 carbon atoms, or from 3 to 6 carbon atoms.

In another non-limiting embodiment, the methyl vinyl ether-maleic acid (MVE-MA) copolymer has a number average molecular weight of between about 50,000 and about 3000,000 Daltons.

In another non-limiting embodiment, the lower alkyl group of vinyl ether-maleic acid copolymer has a number average molecular weight (in Daltons) from about 50,000 to about 100,000, from about 300,000 to about 500,000, from about 500,000 to about 600,000, from about 600,000 to about 900,000, from about 900,000 to about 1200,000, from about 1200,000 to about 1500,000, from about 1500,000 to about 1800,000, from about 1800,000 to about 2100,000, from about 2100,000 to about 2500,000, from about 2500,000 to about 2800,000, or from about 2800,000 to about 3000,000.

In another non-limiting embodiment, the salts of copolymer of alkyl vinyl ether-maleic acid has a specific viscosity of from about 2.5 to about 5.0 when measured as a 1% w/v solution in methyl ethyl ketone (MEK) solution at 25° C.

In another non-limiting embodiment, the lower alkyl group of vinyl ether-maleic acid copolymer has a specific viscosity of from about 2.5 to about 3.0, from about 3.0 to about 3.5, from about 3.5 to about 4.0, from about 4.0 to about 4.5, and from about 4.5 to about 5.0 when measured as a 1% w/v solution in methyl ethyl ketone (MEK) solution at 25° C.

In another non-limiting embodiment, the lower alkyl group of vinyl ether-maleic acid copolymer is present in an amount of from about 5 to about 50 wt. % of the total composition. Further, the lower alkyl group of vinyl ether-maleic acid copolymer is present in an amount of from about 5 to 10 wt. %, from about 10 to about 15 wt. %, from about 15 to about 20 wt. %, from about 20 to 25 wt. %, from about 25 to about 30 wt. %, from about 30 to about 35 wt. %, from about 35 to about 40 wt. %, from about 40 to about 45 wt. %, or from about 45 to about 50 wt. %.

In another non-limiting embodiment, the mucoadhesive composition comprises sodium salt of carboxymethyl cellulose (Na-CMC). The cellulose ether useful herein include the 7H series of sodium carboxymethyl celluloses available from Aqualon having a typical molecular weight of about 700,000 daltons d per their brochure materials. Other examples of useful carboxymethylcellulose include 7H3 SX8F and 7H3 SXF both commercially available from Aqualon/Hercules and CEKOL 30,000P from C.P. Kelco/Noviant/Huber. In some embodiments, the sodium carboxymethyl celluloses used in the described compositions is 7H3SXF.

In another non-limiting embodiment, the present application discloses molecular weight of the sodium carboxymethyl cellulose to be between medium to high. In some embodiments, the medium to high weight average molecular weight is in the range of from about 2000 to about 1,500,000 Daltons. In some embodiments, the medium molecular weight ranges from about 10000 to about 15000 Daltons; or from about 15000 to about 20000 Daltons; or from about 20000 to about 300000; or from about 30000 to about 40000 Daltons; or from about 40000 to about 50000 Daltons; or from about 50000 to about 60000 Daltons; or from about 60000 to about 70000 Daltons; or from about 70000 to about 80000 Daltons. In some embodiments, the high molecular weight ranges from about 80000 to about 90000 Daltons; or from about 90000 to about 100000 Daltons; or from about 100000 to about 200000 Daltons; or from about 200000 to about 300000 Daltons; or from about 300000 to about 400,000 Daltons, or from about 400,000 to about 500,000 Daltons, or from about 500,000 to about 600,000 Daltons, or from about 600,000 to about 700,000 Daltons, or from about 700,000 to about 800,000 Daltons, or from about 800,000 to about 900,000 Daltons, or from about 900,000 to about 1,000,000 Daltons, or from about 1,000,000 to about 1,100,000 Daltons.

In another non-limiting embodiment, the sodium carboxymethyl cellulose (Na-CMC) is present in an amount of from about 5 to about 60 wt. % of the total composition. Further, the sodium carboxymethyl cellulose (Na-CMC) is present in an amount of from about 5 to about 10 wt. %, from about 10 to about 15 wt. %, from about 15 to about 20 wt. %, from about 20 to about 25 wt. %, from about 25 to about 30 wt. %, from about 30 to about 35 wt. %, from about 35 to about 40 wt. %, from about 40 to about 45 wt. %, from about 45 to about 50 wt. %, from about 50 to about 55 wt. %, or from about 55 to about 60 wt. % of the total composition.

In another non-limiting embodiment, the present application provides a strongly swellable, moderately crosslinked PVP (polyvinylpyrrolidone) polymer available under the tradename FlexiThix™ or Polyplasdone™ from Ashland Inc. obtained directly as a fine powder by precipitation polymerization of vinyl pyrrolidone in the presence of a predetermined amount of a multifunctional crosslinking agent and a free radical initiator in an organic solvent. The crosslinked PVP has a Brookfield viscosity of at least about 500 to about 50,000 cps in 4% aqueous solution. The preferred viscosity ranges of the crosslinked PVP in the present application can be varied from about 500 to about 50,000 cps or from about 800 to about 20,000 cps or from about 1000 to about 10,000 cps. The Brookfield viscosity can be measured at 2.5, 5, 10, 12, 20, 30, or 50 RPM and at 25° C.

The crosslinked PVP polymer useful in the practice of the present application can be prepared according to granted U.S. Pat. Nos. 5,073,614, and 5,130,388 assigned to ISP Investments Inc. The teachings of these references are advantageously employed for the purposes of the present application. Further, the references are incorporated herein by reference in their entirety.

An element of the thickening additive composition according to the present application is a thickening agent comprising a strongly swellable, lightly to moderately crosslinked polyvinylpyrrolidone as described in commonly owned U.S. Pat. Nos. 5,312,619 and 5,139,770, the contents of which are hereby incorporated by reference in their entirety. The term “strongly swellable, lightly to moderately crosslinked PVP”, unless otherwise noted, specifically refers to a polymer essentially consisting of lightly-to moderately-crosslinked poly(N-vinyl-2-pyrrolidone) having at least one of the following non-limiting characteristics: (1) an aqueous swelling parameter defined by its gel volume from about 15 mL/g to about 300 mL/g, more particularly from about 15 mL/g to about 250 mL/g, and in other cases from about 15 mL/g to about 150 mL/g, or (2) a Brookfield viscosity of (measured at 5% crosslinked PVP in a liquid carrier comprising water at 25° C.) of at least 2,000 cP, more particularly preferably of at least about 5,000 cP, and in certain cases of at least about 10,000 cP. Reference for these parameter ranges is provided in U.S. Pat. No. 5,073,614 (incorporated herein by reference) and in Shih, J. S., et al. (1995). Synthesis methods for the crosslinked PVP are disclosed in a number of references, including U.S. Pat. Nos. 5,073,614; 5,654,385; and 6,177,068, the contents of which are hereby incorporated by reference. It is appreciated by a polymer scientist skilled in the art that various methods of synthesis are possible, provided that the produced polymer achieves at least one of the above defined parameters.

Crosslinked polyvinylpyrrolidones are commercially available, for example, as Kollidon® CL types from BASF or as Polyplasdone® XL and Flexithix™ type PVPs from Ashland Inc. In another non-limiting embodiment, the present application provides a swellable, crosslinked PVP polymer that can be prepared directly in the form of a fine, white powder by precipitation polymerization of vinylpyrrolidone in the presence of a predetermined amount of a crosslinking agent and a free radical polymerization initiator in an organic solvent, preferably an aliphatic hydrocarbon, e.g., a C₃-C₁₀ saturated, branched or unbranched, cyclic or acyclic aliphatic hydrocarbon, and most preferably, cyclohexane or heptane, or mixtures thereof.

In various embodiments of this application, the crosslinked polymer of vinylpyrrolidone (including copolymers of vinylpyrrolidone and other monomeric materials) in a porous granular or bead form even when wetted and swollen is produced by a process wherein the monomeric material is polymerized with a controlled amount of crosslinking agent in an aqueous solution of electrolyte. The insoluble polymer is formed, and excess monomer is maintained in suspension by mechanical agitation.

In various embodiments of this invention, the crosslinked vinylpyrrolidone polymer provide some rigidity to the hydrated denture adhesive during use preventing structural breakdown of the hydrogel and consequent loss of the same from between gum and denture due to oozing.

In another non-limiting embodiment, the present application provides cross-linked polyvinylpyrrolidone (PVPP) is present in the range of from about 0.5 to about 10 wt. % of the total composition. Accordingly, the cross-linked polyvinylpyrrolidone (PVPP) is present in the range of from about 0.5 to about 1 wt. %, from about 1.0 to about 2 wt. %, from about 2 to about 4 wt. %, from about 4 to about 6 wt. %, from about 6 to about 8 wt. %, or from about 8 to about 10 wt. % to of the total composition.

In another non-limiting embodiment, the present application provides a di-valent (M⁺⁺) cation donor selected from the group consisting of calcium (Ca⁺⁻), strontium (Sr⁺⁺), zinc (Zn⁺⁺), magnesium (Mg⁺⁺), iron (Fe⁺⁺), vanadium (V⁺⁺), chromium (Cr⁺⁺), manganese (Mn⁺⁺), nickel (Ni^{∥}), copper (Cu^{∥}), yttrium (Y^{∥}) and mixtures thereof.

In another non-limiting embodiment, the di-valent (M⁺⁺) cation donor is a salt selected from the group consisting of acetate salt of di-valent cations, carboxylate salt of di-valent cations, gluconate salt of di-valent cations, halide salt of di-valent cations, hydroxide salt of di-valent cations, carbonate salts of di-valent cations, lactate salt of di-valent cations, oxide salt of di-valent cations, phosphate salt of di-valent cations, sulphate salt of di-valent cations, and combinations thereof.

In another non-limiting embodiment, the di-valent (M⁺⁺) cation salt is selected from the group consisting of calcium acetate, calcium carbonate, calcium chloride, calcium 2-ethylbutanoate, calcium gluconate, calcium hydroxide, calcium lactate, calcium oxide, calcium propionate, calcium sulphate, calcium magnesium acetate, magnesium acetate, magnesium chloride, magnesium propionate, zinc chloride, zinc acetate, and combinations thereof.

In another non-limiting embodiment, the di-valent (M⁺⁺) cation donor is present in an amount of from about 1 to about 20 wt. % of the total composition. Further, di-valent (M⁺⁺) cation donor is present in an amount of from about 1 to about 5 wt. %, from about 5 to about 10 wt. %, from about 10 to about 15 wt. %, or from about 15 to about 20 wt. % of the total composition.

In another non-limiting embodiment, the neutralizer is a salt selected from the group consisting of hydroxides of mono-valent (M⁺) cation or divalent (M⁻⁺) cation, phosphate salt of mono-valent (M⁺) cation or di-valent (M⁺⁺) cation, pyrophosphate salt of mono-valent cation or di-valent cation, hydrogen phosphate salt of mono-valent cation or di-valent cation, carbonate or bicarbonate salt of mono-valent cation or di-valent cation, tripolyphosphate or orthometaphosphate salt of mono-valent cation or di-valent cation, hydroxide salts of monovalent cation or divalent cation and combinations thereof.

In various embodiments of this invention, the neutralizer is selected from the group consisting of phosphate salt of mono-valent (M⁺) cation or di-valent (M⁺⁺) cation, pyrophosphate salt of mono-valent cation or di-valent cation, hydrogen phosphate salt of mono-valent cation or di-valent cation, carbonate or bicarbonate salt of mono-valent cation or di-valent cation, orthometaphosphate salt of mono-valent cation or di-valent and combinations thereof.

Accordingly, the neutralizer is selected from the group consisting of sodium hydroxide, sodium orthometaphosphate, sodium hydrogen phosphate, trisodium phosphate, sodium tripolyphosphate, tetrasodium pyrophosphate, sodium hexametaphosphate potassium hydroxide, potassium hydrogen phosphate, and mixtures thereof.

In another non-limiting embodiment, the neutralizer is present in an amount of from about 0.5 to about 20 wt. % of the total composition. Further, neutralizer is present in an amount of from about 0.5 to 5 wt. %, from about 5 to about 10 wt. %, from about 10 to about 15 wt. %, or from about 15 to about 20 wt. % of the total composition.

In another non-limiting embodiment, the present application further comprises about 0.5 to about 60 wt. % of at least one additional cellulose polymer selected from the group consisting of lower alkyl cellulose, hydroxyethylcellulose, hydroxypropyl cellulose, hydroxypropyl methyl cellulose, carboxymethylcellulose, divalent cation modified carboxymethyl cellulose and mixtures thereof.

In another non-limiting embodiment, the present application further comprises about 0.5 to about 5 wt. %, about 5 to about 10 wt. %, about 10 to about 15 wt. %, about 15 to about 20 wt. %, about 20 to about 25 wt. %, about 25 to about 30 wt. %, about 30 to about 35 wt. %, about 35 to about 40 wt. %, about 40 to about 45 wt. %, about 45 to about 50 wt. %, about 50 to about 55 wt. %, about 55 to about 60 wt. % of at least one additional cellulose polymer.

Accordingly, the additional cellulose polymer has a weight average molecular weight in the range from about 2000 to about 1,500,000 Daltons. The additional cellulose polymer has a weight average molecular weight in the range of from about 2000 to about 1,500,000 Daltons. In some embodiments, the medium molecular weight ranges from about 10000 to about 15000 Daltons; or from about 15000 to about 20000 Daltons; or from about 20000 to about 300000; or from about 30000 to about 40000 Daltons; or from about 40000 to about 50000 Daltons; or from about 50000 to about 60000 Daltons; or from about 60000 to about 70000 Daltons; or from about 70000 to about 80000 Daltons. In some embodiments, the high molecular weight ranges from about 80000 to about 90000 Daltons; or from about 90000 to about 100000 Daltons; or from about 100000 to about 200000 Daltons; or from about 200000 to about 300000 Daltons; or from about 300000 to about 400,000 Daltons, or from about 400,000 to 500,000 Daltons, or from about 500,000 to 600,000 Daltons, or from about 600,000 to 700,000 Daltons, or from about 700,000 to 800,000 Daltons, or from about 800,000 to 900,000 Daltons, or from about 900,000 to 1,000,000 Daltons, from about 1,000,000 to 1,100,000 Daltons, from about 1,100,000 to 1,200,000 Daltons, from about 1,200,000 to about 1,300,000 Daltons, from about 1,300,000 to about 1,400,000 Daltons, or from about 1,400,000 to about 1,500,000 Daltons.

In another non-limiting embodiment, the present application provides additional cellulose polymer is carboxymethyl cellulose (CMC) is functionalized with i) at least one mono-valent (M⁺) cation and at least one di-valent (M⁺⁺) cation; or ii) at least one divalent cation. The additional cellulose polymer is carboxymethyl cellulose (CMC) selected from the group consisting of: (i) sodium-calcium carboxymethyl cellulose (Na+-Ca⁺⁺-CMC), (ii) sodium-magnesium carboxymethyl cellulose (Na+-Mg⁺⁺-CMC), (iii) sodium-zinc carboxymethyl cellulose (Na+—Zn++-CMC), (iv) sodium-calcium-zinc carboxymethyl cellulose (Na+-Ca⁺⁺—Zn⁺⁺-CMC), (v) sodium-calcium-magnesium carboxymethyl cellulose (Na+-Ca⁺⁺-Mg++-CMC), (vi) sodium-magnesium-zinc cellulose (Na+-Mg⁺⁺—Zn⁺⁺-CMC), (vii) sodium-tin-carboxymethyl cellulose (Na+—Sn⁺⁺-CMC), (viii) sodium-calcium-tin-carboxymethyl cellulose (Na+-Ca⁺⁺—Sn⁺⁺-CMC), (ix) sodium-magnesium-tin-carboxymethyl cellulose (Na+-Mg⁺⁺—Sn⁺⁺-CMC), or (x) sodium-zinc-tin-carboxymethyl cellulose (Na+—Zn⁺⁺—Sn⁺⁺-CMC), (xi) calcium-carboxymethyl cellulose (Ca⁺⁺-CMC), (xii) magnesium-carboxymethyl cellulose (Mg⁺⁺-CMC), (xiii) zinc-carboxymethyl cellulose (Zn⁺⁺-CMC), or (xiv) tin-carboxymethyl cellulose (Sn⁺⁺-CMC).

In another non-limiting embodiment, the present mucoadhesive composition provides one more oral care acceptable ingredient selected from the group consisting of adhesion promoters, additional modified cellulose ethers, anti-caking agents, anti-fungal agents, anti-microbial agents, anti-gingivitis agents, anesthetic agents, antioxidants, anti-biotics, anti-inflammatory agents, binder, buffers, colors, cooling agents, dentinal desensitizing agents, dispersants, enzymes, emollients, flavors, fillers, fragrances, gelling agent, humectants, hydrophilic non-oil components, oil carriers, ooze control polymers, preservatives, pigments, plasticizers, pain relieving agents, sweeteners, thickening agents, viscosity modifiers, vehicles, surfactants, stabilizers, sensates and mixtures thereof.

In another embodiment, the oral care acceptable ingredient is present in an amount from about 0.001 to about 90 wt. % of the total composition. Further, the oral care ingredient is present in an amount of from about 0.001 to about 10 wt. %, from about 10 to about 20 wt. %, from about 20 to about 30 wt. %, from about 30 to about 40 wt. %, from about 40 to about 50 wt. %, from about 50 to about 60 wt. %, from about 60 to about 70 wt. %, from about 70 to about 80 wt. %, from about 80 to about 90 wt. % of the total composition.

Non-limiting examples of emollients include hydrophobic oil or hydrophilic non-oil components.

Accordingly, the hydrophobic oil or hydrophilic non-oil components can be selected from the group consisting of liquid petrolatum, petrolatum, mineral oil, glycerin, natural and synthetic oils, fats, silicone and silicone derivatives, polyvinylacetate, polyethylene glycol, propylene glycol, polypropylene glycol, poly(ethylene oxide-propylene oxide) copolymer, diethylene glycol, triethylene glycol, sorbitol, water, orally acceptable surfactant and mixtures thereof, natural and synthetic waxes such as animal waxes like beeswax, lanolin and shellac, hydrocarbons, hydrocarbon derivatives, vegetable oil waxes such as carnauba, candela and bayberry wax, vegetable oils such as caprylic/capric triglycerides, vegetable oils such as corn, sunflower, soy bean, castor, palm, coconut, olive, and rapeseed oil or mixtures thereof, and animal oil such as fish oil and oleic acid, and mixtures thereof. Non limiting examples of mineral oil include maleated avocado oils, maleated coconut oils, maleated corn oils, maleated cottonseed oils, maleated jojoba oils, maleated linseed oils, maleated nut oils, maleated olive oils, maleated palm oils, maleated raisin oils, maleated rapeseed oils, maleated safflower oils, maleated sesame oils, maleated soybean oils, maleated squash oils, maleated sunflower oils, maleated almond oils, maleated canola oils, maleated flaxseed oils, maleated grapeseed oils, maleated palm oils, maleated palm kernel oils, maleated peanut oils, maleated walnut oils.

In another non-limiting embodiment, the mineral oil is functionalized or unfunctionalized maleated soybean oil, wherein the functionalization includes hydrophilic or hydrophobic moieties.

In some embodiments, the hydrophobic oil or hydrophilic non-oil components based carriers is/are present in suitable amounts ranging from about 10 wt. % to about 20 wt. %, or from about 20 wt. % to about 30 wt. %, or from about 30 wt. % to about 40 wt. %, or from about 40 wt. % to about 50 wt. % or from about 50 wt. % to about 60 wt. % or from about 60 wt. % to about 75 wt. % based on the total weight of the mucoadhesive composition including denture adhesive composition.

Non-limiting examples of colorants are selected from the group consisting of talc, mica, magnesium carbonate, calcium carbonate, magnesium silicate, aluminum magnesium silicate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder, methacrylate powder, polystyrene powder, silk powder, crystalline cellulose, starch, titanated mica, iron oxide titanated mica, bismuth oxychloride, and mixtures thereof.

Non-limiting examples of flavoring agent include (i) synthetic flavoring liquid and/or oil derived from plants leaves, flowers, and fruits, including vanillin, sage, marjoram, parsley oil, spearmint oil, cinnamon oil, oil of wintergreen (methyl salicylate), peppermint oils, clove oil, bay oil, anise oil, and eucalyptus oil; (ii) artificial, natural, or synthetic fruit flavor extracted from lemon, orange, banana, grape, lime, apricot, and grapefruit; (iii) fruit essence prepared from apple, strawberry, cherry, orange, and pineapple; (iv) bean and nut derived flavors including coffee, cocoa, cola, peanut, almond; (v) flavors adsorbed onto a hydrophilic matrix such as “spray-dried” flavors, and (vi) encapsulated flavors.

The amount of flavoring agent flavorant employed is normally a matter of preference subject to such factors as flavor type and strength of flavor desired. Flavorants may be present in amounts up to about 4%, in one embodiment about 0.05% to about 3.0%, in another embodiment about 0.8% to about 2.5%, by weight of the total composition.

Non-limiting examples of antibacterial compounds used in the present composition can be selected from the group consisting of halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), biguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, Zinc Chloride, Zinc Lactate, Zinc Sulfate, stannous salts, copper salts, iron salts), sanguinarine, propolis a stannous fluoride, sodium fluoride, potassium fluoride, sodium monofluorophosphate, sodium fluorosilicate, ammonium fluorosilicate, amine fluoride and ammonium fluoride. The anti-inflammatory compounds of the present application can be selected from the group including but not limited to triamcinolone (trade name: Kenalog), fluocinonide (trade name: Vanos), dexamethasone (trade name: decadron), herpes, amlexanox (aphthasol), ketorolac, flurbiprofen, ibuprofen, naproxen, indomethacin, aspirin, ketoprofen, piroxicam and meclofenamic acid.

Non-limiting examples of pain relieving compounds of the present application can be selected from the group including but not limited to benzocaine, lidocaine, procaine, prilocaine, mepivacaine, aspirin, ibuprofen, diclofenac, and methyl salicylate.

Non-limiting examples of anti-oxidant compounds of the present application can be selected from the group including but not limited to vitamin E, ascorbic acid, uric acid, carotenoids, vitamin A, flavonoids, polyphenols, herbal antioxidants, melatonin, amino-indoles, lipoic acids, caffeic acid, beta-carotene, ellagic acid, epicatechin, epicatechin gallate, ferulic add, genistein, kojic acid, alpha-lipoic acid, lycopene, resveratrol, resorcinol, rosmarinic acid, silibinin, theaflavin, tocopherols, tocotrienols, trolox and ubiquinone-10.

Non-limiting examples of enzymes of the present application can be selected from the group including but not limited to Proteases; papain, bromelain, chymotrypsin, ficin and alcalase; Carbohydrases: glucoamylase, alpha-amylase, beta-amylase, dextranase and mutanas; Lipases: plant lipase, gastric lipase and pancreatic lipase and Glucoamylase, a Saccharifying glucoamylase of Aspergillus niger origin.

Non-limiting examples of cooling agents of the present application can be selected from the group including but not limited to menthol, menthyl lactate, monomenthyl succinate, menthol ethylene glycol carbonate, menthol propylene glycol carbonate, menthone glycerol ketal, 3-(l-menthoxy) propane-1,2-diol, (−)-isopulegol, WS-3 1 [N-ethyl-p-menthane-3-carboxamide], WS-23 (2-isopropyl-N-2,3-trimethylbutyramide), and WS-5 [ethyl 3p-menthane-3-carboxamido)acetate].

The cooling agents of the present application can be selected from the group including but not limited to acesulfame K, aspartame, neotame, saccharin, sucralose, stevia, advantame cyclamate, sorbitol, xylitol and erythritol.

In some embodiments, an antibacterial, an anti-inflammatory, a pain reliving agent, an antioxidant, an enzyme, a flavor, a cooling agent, and a sweetener is/are present in suitable amounts ranging from about 0.01 wt. % to about 0.1 wt. %, or from about 0.1 wt. % to about 1 wt. %, or from about 1 wt. % to about 10 wt. %, or from about 10 wt. % to about 20 wt. % based on the total weight of the mucoadhesive composition including denture adhesive and denture fixative composition.

According to another embodiment of the present application, it is contemplated to employ at least one excipient. Suitable excipients for the purposes of this application are selected from the group consisting of a preservative, a flavoring agent, a colorant, a sweetener, a plasticizer, a binder, a thickener, vehicles, colorant, carrier, flavor, fragrance, sensate, arid mixtures thereof.

In another non-limiting embodiment, the present application discloses making mucoadhesive composition and of using such formulations for sustained release of active ingredients selected from the group including but not limited to an antibacterial, an anti-inflammatory, a pain reliving agent, an antioxidant, an enzyme, a flavor, a cooling agent and a sweetener.

In one embodiment, the mucoadhesive composition is a an oral gel, an oral ointments, buccal composition, a sublingual composition, a palatal composition, and a denture adhesive composition in the form of a dentifrice, denture cleanser, chewing gum, lozenge, mouth, dental implement tooth powder, topical oral gel, denture product, dissolvable film, strip, oral tablet, wafer, chewing gum.

In one embodiment, it is contemplated to use a material that is suitable for mucoadhesive composition including denture adhesive, particularly, it is safe and palatable at relevant concentrations for use in a hygiene composition, such as, paste, cream, gel, thermoplastic solid, hydrogel or combinations thereof.

In another non-limiting embodiment, the present mucoadhesive composition has a pH of from about 5 to about 8.

In another non-limiting embodiment, the present application provides a method of in situ curing of mucoadhesive composition comprising: (a) from about 2 to about 60 wt. % of non-neutralized diacidic form of lower alkyl vinyl ether-maleic acid copolymer, (b) from about 5 to about 60 wt. % of a sodium carboxymethylcellulose (Na-CMC) polymer, (c) from about 0.5 to about 10 wt. % of water-insoluble, water-swellable, cross-linked polyvinylpyrrolidone (PVPP) polymer, (d) from about 0.5 to about 25 wt. % of at least one di-valent (M⁺⁺) cation donor, (e) from about 0.5 to about 25 wt. % of at least one neutralizer, and (f) from about 0.001 to about 90 wt. % of at least one orally acceptable carrier and/or oral care ingredient; the method comprising the steps of: (a) providing from about 0.1 to about 99.9 wt. % of mucoadhesive composition at the application site, (b) releasing di-valent (M⁺⁺) cation in situ by hydration of di-valent (M⁺⁺) cation donor in the presence of or by introduction of a hydrating agent, (c) enabling di-valent cation mediated crosslinking of the lower alkyl vinyl ether-maleic acid copolymer, (d) forming an interpenetrating polymer network (IPN) of lower alkyl vinyl ether-maleic acid copolymer and carboxymethylcellulose, and (e) obtaining an in situ activated and cured mucoadhesive composition.

Accordingly, the hydration agent is water or saliva.

In another non-limiting embodiment, the mucoadhesive composition is a denture fixative composition.

Further, certain aspects of the present application are illustrated in detail by way of the following examples. The examples are given herein for illustration of the application and are not intended to be limiting thereof.

### EXAMPLES

### Example 1: Oil-Based Mucoadhesive Formulation

200 g batch of standard oil-based mucoadhesive formulation was prepared in a KitchenAid planetary mixer equipped with a digitally controlled constant temperature heating jacket. The heating jacket was set to maintain a constant temperature of 194° F. (90° C.). Components required to make mucoadhesive cream were added as per Table 1. Petrolatum and white mineral oil were charged into the heated bowl of the KitchenAid mixer and mixed at low speed for 10 minutes till petrolatum completely melted and produced a homogeneous transparent liquid. Sodium Carboxymethyl cellulose was charged into the KitchenAid and mixed vigorously with the oils for 30 minutes till uniformly dispersed. If applicable, other cellulose ether(s) were charged to the mixing bowl of the KitchenAid and mixed vigorously for another 10 minutes. Poly (MVE/MA) diacid polymer powder was then charged into the KitchenAid and mixed at medium speed for another 10 minutes to yield a uniform consistency paste. Neutralizer, divalent cation doner and ooze control agents, as shown in Table 1, were charged to the mixing bowl of the KithenAid then and mixed vigorously for 30 minutes till a uniform paste was obtained. Suitable color and other ingredients were added and mixed till uniformly dispersed. The heat was turned off and the mucoadhesive cream was allowed to cool down to room temperature over a period of 2 hours under continuous stirring at medium speed. After cooling down to room temperature, 50 g mucoadhesive sample was packed in proper dispensing tube and heat sealed. Remaining mucoadhesive was packed in clear glass jars and used for storage stability studies.

### Example 2: Mucoadhesive Test Method

Adhesion forces and relative mucoadhesive film thickness were recorded on TA.XT Plus texture analyzer instruments from Stable Microsystems Texture Technologies Corp., equipped with a 50 Kg load cell and interfaced with a PC running Exponent software version 6.1.11.0. The texture analyzer instruments were equipped with a custom-built denture shaped plexiglass probe-fixture assembly. Artificial saliva infusion between the walls of the plexiglass probe-fixture assembly was achieved using peristaltic pumps. Real-time time-lapse images of mucoadhesive creams undergoing adhesion performance evaluation on the texture analyzer instruments were recorded using a Canon EOS 5d Mark IV Digital SLR camera.

Sample tubes containing mucoadhesive creams were vented for trapped air and with the cap on, squished several times to homogenize the contents inside the tube. The first 100 mg of material coming out of the tube was always discarded.

Three nearly identical strips of mucoadhesive cream were weighed out into the denture shaped cavity of the bottom plexiglass fixture on an analytical balance. Total mass of mucoadhesive cream applied to the bottom fixture for each test was 2. 0 (+0.1) g. The bottom fixture was mounted on to the texture analyzer instrument and the top probe was moved down into the cavity of the bottom fixture to uniformly spread the mucoadhesive cream and fill the bottom half of the cavity under a precise compression force of 4.5 Kg. Infusion of artificial saliva was initiated. For the entire duration of the experiment, the mucoadhesive cream remained in contact and submerged under a thin level of artificial saliva which was continuously refreshed at a flow rate of 1 ml/minute. The 7-hour test sequence was initiated once the mucoadhesive cream layer was fully covered with artificial saliva. Adhesion force (in Newtons) at instances of mastication and thickness of mucoadhesive film between the top and the bottom probe (in mm) were continuously measured for the duration of the experiment. Each sample of prepared mucoadhesive formulations was subjected to four consecutive experimental runs. The average plots from 4 runs for each sample were compared for adhesion performance, and thickness variation. Total adhesion for each sample was calculated as the area under the curve for the average plot of the 4 consecutive runs for each sample (Examples 1, 4, 7-9, 14-18, 20 and 21) and reported in Table 1.

TABLE 1
Mucoadhesive formulations and Total adhesion

| | | Control | Exp. 1 | Exp. 4 | Exp. 7 | Exp. 8 | Exp. 9 | Exp. 14 |
|---|---|---|---|---|---|---|---|---|
| Component | Name | (wt %) | (wt %) | (wt %) | (wt %) | (wt %) | (wt %) | (wt %) |
| Oil carriers | Petrolatum | 20.00 | 20.00 | 20.00 | 20.00 | 20.00 | 20.00 | 20.00 |
| | White mineral oil | 20.00 | 20.00 | 20.00 | 20.00 | 20.00 | 20.00 | 20.00 |
| Secondary | Aqualon ™ CMC 7H3SXF | 27.00 | 51.65 | 31.50 | 43.20 | 39.20 | 33.80 | 37.00 |
| adhesive | (Sodium carboxymethyl cellulose) | | | | | | | |
| polymer | | | | | | | | |
| Primary | Gantrez ™ MS-955 | 33.00 | — | — | — | — | — | — |
| adhesive | (Na/Ca salt of PVM/MA polymer) | | | | | | | |
| polymer | (PVM/MA diacid polymer) | — | 5.00 | 15.00 | 10.00 | 10.00 | 10.00 | 10.00 |
| Neutralizer | Disodium hydrogen phosphate, | — | 0.85 | 3.50 | 1.80 | 1.80 | 1.80 | 6.00 |
| | dihydrate | | | | | | | |
| | Tetrasodium pyrophosphate | — | — | — | — | — | — | — |
| | Sodium tripolyphosphate | — | — | — | — | — | — | — |
| Divalent | Calcium chloride | — | 2.50 | 10.00 | 5.00 | — | — | 5.00 |
| cation donor | Calcium carbonate | — | — | — | — | 9.00 | — | — |
| | Calcium-L-lactate | — | — | — | — | — | 14.40 | — |
| Ooze control | Polyplasdone ™ XL-10 | — | — | — | — | — | — | 2.00 |
| agent | (crosslinked PVP) | | | | | | | |
| | Total (wt %) | 100.00 | 100.00 | 100.00 | 100.00 | 100.00 | 100.00 | 100.00 |
| | Total adhesion (Newtons) | 35997 | 48753 | 41408 | 49998 | 25551 | 32930 | 45302 |

| | | Exp. 15 | Exp. 17 | Exp. 18 | Exp. 20 | Exp. 21 |
|---|---|---|---|---|---|---|
| Component | Name | (wt %) | (wt %) | (wt %) | (wt %) | (wt %) |
| Oil carriers | Petrolatum | 20.00 | 20.00 | 20.00 | 20.00 | 20.00 |
| | White mineral oil | 20.00 | 20.00 | 20.00 | 20.00 | 20.00 |
| Secondary | Aqualon ™ CMC 7H3SXF | 38.00 | 40.00 | 41.00 | 40.00 | 41.00 |
| adhesive | (Sodium carboxymethyl cellulose) | | | | | |
| polymer | | | | | | |
| Primary | Gantrez ™ MS-955 | — | — | — | — | — |
| adhesive | (Na/Ca salt of PVM/MA polymer) | | | | | |
| polymer | (PVM/MA diacid polymer) | 10.00 | 10.00 | 10.00 | 10.00 | 10.00 |
| Neutralizer | Disodium hydrogen phosphate, | 6.00 | — | — | — | — |
| | dihydrate | | | | | |
| | Tetrasodium pyrophosphate | — | 3.00 | 3.00 | — | — |
| | Sodium tripolyphosphate | — | — | — | 3.00 | 3.00 |
| Divalent | Calcium chloride | 5.00 | 5.00 | 5.00 | 5.00 | 5.00 |
| cation donor | Calcium carbonate | — | — | — | — | — |
| | Calcium-L-lactate | — | — | — | — | — |
| Ooze control | Polyplasdone ™ XL-10 | 1.00 | 2.00 | 1.00 | 2.00 | 1.00 |
| agent | (crosslinked PVP) | | | | | |
| | Total (wt %) | 100.00 | 100.00 | 100.00 | 100.00 | 100.00 |
| | Total adhesion (Newtons) | 42364 | 45262 | 45532 | 49941 | 51834 |

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. The embodiments of the invention in which an exclusive property or privilege is claimed are defined as follows.

What is claimed is:

1. A mucoadhesive composition, comprising:
(a) from about 2 to about 60 wt. % of non-neutralized diacidic form of lower alkyl vinyl ether-maleic acid copolymer,
(b) from about 5 to about 60 wt. % of a sodium carboxymethylcellulose (Na-CMC) polymer,
(c) from about 0.5 to about 10 wt. % of water-insoluble, water-swellable, cross-linked polyvinylpyrrolidone (PVPP) polymer,
(d) from about 0.5 to about 25 wt. % of at least one di-valent (M⁺⁺) cation donor,
(e) from about 0.5 to about 25 wt. % of at least one neutralizer, and
(f) from about 0.001 to about 90 wt. % of at least one oral care ingredient.

2. The mucoadhesive composition according to claim 1, wherein the lower alkyl group of vinyl ether-maleic acid copolymer has from about 1 to about 12 carbon atoms.

3. (canceled)

4. The mucoadhesive composition according to claim 2, wherein the lower alkyl vinyl ether-maleic acid copolymer is selected from the group consisting of methoxy ethylene maleic acid copolymer, ethyl vinyl ether maleic acid copolymer, propyl vinyl ether maleic acid copolymer, isobutyl vinyl ether maleic acid copolymer and mixtures thereof;
the average molecular weight of the lower alkyl vinyl ether-maleic acid copolymer is in the range of from about 50,000 to about 3,000,000 Daltons;
the lower alkyl vinyl ether-maleic acid copolymer has a specific viscosity of from about 2.5 to about 5.0% when measured as a 1% w/v solution in methyl ethyl ketone (MEK) solution at 25° C.; and
the lower alkyl vinyl ether-maleic acid copolymer is present in an amount of from about 5 to about 50 wt. % of the total composition.

5. (canceled)

6. (canceled)

7. (canceled)

8. The mucoadhesive composition according to claim 1, wherein the sodium carboxymethylcellulose has a number average molecular weight is in the range of from about 50,000 to about 1,000,000 Daltons;
or
wherein the sodium carboxymethylcellulose is present in the range of from about 5 to about 60 wt. % of the total composition.

9. (canceled)

10. The mucoadhesive composition according to claim 1, wherein the cross-linked polyvinylpyrrolidone (PVPP) has a gel volume of from about 15 to about 300 ml/g; and
the cross-linked polyvinylpyrrolidone (PVPP) is present in the range of from about 0.5 to about 10 wt. % of the total composition.

11. (canceled)

12. The mucoadhesive composition according to claim 1, wherein the di-valent (M⁺⁺) cation donor is selected from the group consisting salts of calcium (Ca⁺⁺), strontium (Sr⁺⁺), zinc (Zn⁺⁺), magnesium (Mg⁺⁺), iron (Fe⁺⁺), vanadium (V⁺⁺), chromium (Cr⁺⁺), manganese (Mn⁺⁺), nickel (Ni⁺⁺), copper (Cu⁺⁺), yttrium (Y⁺⁺), and mixtures thereof;
or
wherein the di-valent (M⁺⁺) cation donor is a salt selected from the group consisting of acetate salt of di-valent cations, carboxylate salt of di-valent cations, gluconate salt of di-valent cations, halide salt of di-valent cations, hydroxide salt of di-valent cations, lactate salt of di-valent cations, oxide salt of di-valent cations, phosphate salt of di-valent cations, sulphate salt of di-valent cations, and combinations thereof.

13. (canceled)

14. The mucoadhesive composition according to claim 1, wherein the di-valent (M⁺⁺) cation salt is selected from the group consisting of calcium acetate, calcium carbonate, calcium chloride, calcium 2-ethylbutanoate, calcium gluconate, calcium hydroxide, calcium lactate, calcium oxide, calcium propionate, calcium sulphate, calcium magnesium acetate, magnesium acetate, magnesium chloride, magnesium propionate, zinc sulphate, zinc chloride, zinc acetate, and combinations thereof;
and
the di-valent (M⁺⁺) cation donor is present in amount from about 1 to about 20 wt. % of the total composition.

15. (canceled)

16. The mucoadhesive composition according to claim 1, wherein the neutralizer is a salt of mono-valent (M⁺) cation or a di-valent (M⁺⁺) cation selected from the group consisting of calcium (Ca⁺⁺), sodium (Na⁺), strontium (Sr⁺⁺), zinc (Zn⁺⁺), magnesium (Mg⁺⁺), iron (Fe⁺⁺), potassium (K⁺), vanadium (V⁺⁺), chromium (Cr⁺⁺), manganese (Mn⁺⁺), nickel (Ni⁺⁺), copper (Cu⁺⁺), yttrium (Y⁺⁺), and mixtures thereof;
or
wherein the neutralizer is selected from the group consisting of hydroxides of mono-valent (M⁺) cation or divalent (M⁺⁺) cation, phosphate salt of mono-valent (M⁺) cation or di-valent (M⁺⁺) cation, pyrophosphate salt of mono-valent cation or di-valent cation, hydrogen phosphate salt of mono-valent cation or di-valent cation, carbonate or bicarbonate salt of mono-valent cation or di-valent cation, tripolyphosphate or orthometaphosphate salt of mono-valent cation or di-valent cation, hydroxide salts of monovalent cation or divalent cation and combinations thereof.

17. (canceled)

18. The mucoadhesive composition according to claim 1, wherein the neutralizer is selected from the group consisting of sodium hydroxide, sodium orthometaphosphate, sodium hydrogen phosphate, trisodium phosphate, sodium tripolyphosphate, tetrasodium pyrophosphate, sodium hexametaphosphate potassium hydroxide, potassium hydrogen phosphate, and mixtures thereof;
and
wherein the neutralizer is present in an amount of from about 0.5 to about 20 wt. % of the total composition.

19. (canceled)

20. The mucoadhesive composition according to claim 1, wherein the composition further comprises about 0.5 to about 60 wt. % of at least one additional cellulose polymer selected from the group consisting of lower alkyl cellulose, hydroxyethylcellulose, hydroxypropyl cellulose, hydroxypropyl methyl cellulose, carboxymethylcellulose, divalent cation modified carboxymethyl cellulose and mixtures thereof; the additional cellulose polymer has a weight average molecular weight in the range from about 2000 to about 1,500,000 Daltons.

21. (canceled)

22. The mucoadhesive composition according to claim 20, wherein the divalent cation modified carboxymethyl cellulose (CMC) is functionalized with (i) at least one mono-valent (M⁺) cation and at least one di-valent (M⁺⁺) cation; or (ii) at least one divalent cation selected from the group consisting of:
(i) sodium-calcium carboxymethyl cellulose (Na⁺—Ca⁺⁺-CMC),
(ii) sodium-magnesium carboxymethyl cellulose (Na⁺—Mg⁺⁺-CMC),
(iii) sodium-zinc carboxymethyl cellulose (Na⁺—Zn⁺⁺-CMC),
(iv) sodium-calcium-zinc carboxymethyl cellulose (Na⁺—Ca⁺⁺—Zn⁺⁺-CMC),
(v) sodium-calcium-magnesium carboxymethyl cellulose (Na⁺—Ca⁺⁺—Mg⁺⁺-CMC),
(vi) sodium-magnesium-zinc carboxymethyl cellulose (Na⁺—Mg⁺⁺—Zn⁺⁺-CMC),
(vii) sodium-tin-carboxymethyl cellulose (Na⁺—Sn⁺⁺-CMC),
(viii) sodium-calcium-tin-carboxymethyl cellulose (Na⁺—Ca⁺⁺—Sn⁺⁺-CMC),
(ix) sodium-magnesium-tin-carboxymethyl cellulose (Na⁺—Mg⁺⁺—Sn⁺⁺-CMC),
(x) sodium-zinc-tin-carboxymethyl cellulose (Na⁺—Zn⁺⁺—Sn⁺⁺-CMC),
(xi) calcium-carboxymethyl cellulose (Ca⁺⁺-CMC),
(xii) magnesium-carboxymethyl cellulose (Mg⁺⁺-CMC),
(xiii) zinc-carboxymethyl cellulose (Zn⁺⁺-CMC), or
(xiv) tin-carboxymethyl cellulose (Sn⁺⁺-CMC).

23. The mucoadhesive composition according to claim 1, wherein the one or more oral care acceptable ingredient is selected from the group consisting of adhesion promoters, additional modified cellulose ethers, anti-caking agents, anti-fungal agents, anti-microbial agents, anti-gingivitis agents, anesthetic agents, antioxidants, anti-biotics, anti-inflammatory agents, binder, buffers, colors, cooling agents, dentinal desensitizing agents, dispersants, enzymes, emollients, flavors, fillers, fragrances, gelling agent, humectants, hydrophilic non-oil components, oil carriers, ooze control polymers, preservatives, pigments, plasticizers, pain relieving agents, sweeteners, thickening agents, viscosity modifiers, vehicles, surfactants, stabilizers, sensates and mixtures thereof;
or
wherein the one or more oral care acceptable ingredient is present in an amount from about 0.001 to about 90 wt. % of the total composition.

24. (canceled)

25. (canceled)

26. (canceled)

27. (canceled)

28. The mucoadhesive composition according to claim 1, wherein the composition has a pH of from about 5 to about 8;
or
wherein the composition is formulated into an ointment, a solid, a semisolid, an emulsion, a powder, a paste, a dispersion, a gel, a patch.

29. (canceled)

30. A method of in situ curing of mucoadhesive composition of claim 1, comprising the steps of:
(a) providing from about 0.01 to about 99.9 wt. % of mucoadhesive composition of claim 1 at the application site,
(b) releasing di-valent (M⁺⁺) cation in situ by hydration of di-valent (M⁺⁺) cation donor in the presence of or by introduction of a hydrating agent,
(c) enabling di-valent cation mediated crosslinking of the lower alkyl vinyl ether-maleic acid copolymer,
(d) forming an interpenetrating polymer network (IPN) of lower alkyl vinyl ether-maleic acid copolymer and carboxymethylcellulose, and
(e) obtaining an in situ activated and cured mucoadhesive composition of claim 1.

31. The method of in situ curing of mucoadhesive composition of claim 30, wherein the hydrating agent is water or saliva;
or
wherein the mucoadhesive composition is a denture fixative or denture adhesive composition.

32. (canceled)
